(19) United States
(12) Patent Application Publication
(10) Pub. No.: US 2011/0184057 A1
(43) Pub. Date: Jul. 28, 2011

(54) PARENTERAL TREATMENT WITH STATINS

(76) Inventors: Gregory R. Mundy, San Antonio, TX (US); Helen Mundy, San Antonio, TX (US); Tadmor Shalon, Palo Alto, CA (US); Samuel P. Sawan, Tyngsboro, MA (US)

(21) Appl. No.: 13/014,170

(22) Filed: Jan. 26, 2011

Related U.S. Application Data
(63) Continuation of application No. PCT/US09/04304, filed on Jul. 24, 2009.
(60) Provisional application No. 61/129,899, filed on Jul. 28, 2008.

(51) Int. Cl. A61K 31/351; A61K 31/222; A61P 35/00; A61P 25/28

(52) U.S. Cl. 514/460; 514/548

(57) ABSTRACT

Cancer patients, patients with cognitive dysfunction, or patients with pulmonary hypertension are treated with parenterally administered therapeutic doses of statins. Particularly, transdermal, injection employing liquid infusion or particles are employed. For cancer, the statins may be used by themselves or in conjunction with a chemotherapeutic regimen. Liver cancer is treated orally with super doses of statins.

### CROSS-REFERENCE TO RELATED APPLICATIONS

This application claims the benefit under 35 U.S.C. §119(e)(1) of U.S. Provisional Patent Application No. 61/129,899, filed 28 Jul. 2008, which application is incorporated herein by reference in its entirety.

### INTRODUCTION

1. Field of the Invention

The field of the invention is parenteral treatment with statins to treat abnormal cellular proliferation.

2. Background of the Invention

Statins are known to be HMG-CoA Reductase inhibitors. As such they have been found to be effective in reducing cholesterol levels in blood. For this purpose, the statins are taken orally and it is found that a substantial proportion of the statin dosage is taken up by the liver. The statins have also been reported to induce the production of a plurality of bone morphogenetic proteins. There are reports that the statins are able to induce bone formation in test animals, where the protocols are indicative of behavior in humans.

The statins are widely used for controlling cholesterol in humans. There have been many studies to investigate whether the statins have a positive or negative effect on individuals. These studies have included apparently healthy individuals where the subsequent incidence of cancer has been monitored. Buhaescu and Izzedine 2007 Clin Biochem 40, 575-84 suggest that statins may have a therapeutic effect in a variety of diseases, both neoplastic and neurological. Friis and Olsen 2006 Cancer Invest 24, 413-24 indicate that based on clinical and observational studies the effect of statins on tumors is inconclusive. Katz 2005 Nat Clin Pract Oncol 2, 82-9, suggests that statins may be used as an auxiliary treatment to cancer therapeutics. Cafforio, et al., 2005 Carcinogenesis 26, 883-91 based on a study with cell lines suggest that statins may have a cytotoxic effect on certain tumors by promoting apoptosis. Graaf, et al., 2004 Cancer Treat Rev 30, 609-41 question whether Ras farnesylation is the mechanism by which statins exert their anti-tumor effect and encourage the use of statins with other chemotherapeutic agents. Boucher et al 2006 Microvasc Res 71, 91-102 report the existence of evidence that statin drug use results in a significant reduction in cancer incidence. Other studies have compared the effect of the use or the absence of use of statins on patients with cancer. A sampling of these reports includes: Coogan, et al., Epidemiology 2007 where no association between statin use and cancer occurrence was reported; Fries, et al., Int J Cancer 2005 114:643-7, where a slightly reduced incidence of cancer was reported; Coogan, et al., J Natl Cancer Int 2007 99:32-40, where a statistically lower incidence of Stage 1V was observed with statins; Plotz, et al., J Natl Cancer Int 2006 98:1819-25, who reported a reduced risk of advanced prostate cancer; Setojushi, et al., Circulation 2007 120:833-43, who reported no effect on colorectal, lung or breast cancer; Browning and Martin, Int J Cancer 2007 120:833-43, who reported that there was no apparent short term risk of cancer due to taking statins; Freeman, et al., J Natl Cancer Int 2006 98:1528-46, who reported that statins do not prevent melanoma; and Poynter et al., 2005 NEJM 352:21 who reported that statins appeared to have a beneficial effect with colon cancer patients.

The following patents and patent applications relate to the use of statins in cancer treatment and a short commentary concerning these references is provided:
U.S. Pat. No. 6,534,540 Statins for inhibiting mitochondrial respiration as part of cancer treatment.
US2005/0084489 Integrin αᵥβ₃ antagonist with statin or bisphosphonate (paper); suggests injection at the site
US2005/0058725A1 Anti-metalloproteinases and anti-neoplastic agents. Does not mention statins, but does mention bisphosphonates
US2004/0092565A 1 Prior cystine treatment, with administration of statin and Cox-2 inhibitor immediately prior to chemotherapy (paper)
US2002/0173538A1 Sensitize cancer cells with statins (oral, animal studies)
WO06/024026A2 Tumors having activated myc are responsive to treatment with statins
WO05/042710A1 LFA-1 inhibiting statins for preventing EBV or other viral associated tumors
WO03/022268A1 Prior to chemotherapy, treat with statin and Cox-2 inhibitor

For the most part, the studies of the effect of statins on cancer have been concerned with cell lines or anecdotal evidence or observations associated with other studies. In the case of the treatment of humans, the studies have not been controlled for determining the effect of statins on disease progression and there has been no effort to define effective methods of administration of the statins, by themselves or in conjunction with other drugs.

Etminian, et al., 2003 Pharmacotherapy 23:726-30 report based on a meta-analysis that statins appear to have a beneficial effect on cognitive function and reduce the risk of Alzheimer's disease. Other suggestions that statins may have a salutary effect in relation to cognitive function include Green, et al., Neurobiol Aging 2002 23:S273-4; Hajjar, et al., J Gerono A Biol Sci Med 2002 57:408-14 and Refolo, et al., Neurbiol Dis 2000 7:321-31.

Another neurological disorder is neurofibromatosis Type 1, where statins may find a role in the treatment of the disorder. See, for example, Koth, et al., Neuropsychol Dev Cogn Sect C Child Neuropsychol 2000 6:185-195; Ozonoff, Am. J. Med Genet. 1999 89:45-52 and Weidong Li, et al.,

That statins are found to affect BMP activity is reported by Hu, et al., Biochem Biophys Res Commun 2006 339:59-64, where simvastatin enhances steady-state BMPR2 and expression in microvascular endothelial cells; and Maeda, et al., J Cell Biochem 2004 92:458-71, who report that simvastatin increases expression of BMP2 in osteoblastic cells; while Izumo, et al. Methods Find Exp Clin Pharmacol 2001 23:389-94, report that simvastatin and fluvastatin, but not pravastatin induce osteoblast calcification in a BMP independent manner.

Statins are very attractive drugs as they are known to be safe with chronic use. They also have a number of salutary effects which are related to dosage and manner of administration. It is therefore of interest to determine whether statins may find application in the treatment of cancers, by themselves or in conjunction with other anti-cancer agents.

### REFERENCE TO RELATED PUBLICATIONS

Hindler, et al. 2006 Oncologist 11:306-15 provides an extensive overview of the role of statins and cancer with a broad spectrum of references. Dolak and Jozkowicz 2005 Curr Cancer Drug Targets 5:579-94 describes statin anti-angiogenesis activity.

### SUMMARY OF THE INVENTION

Therapeutically effective administration of statins for the treatment of neoplasia and neurological diseases is provided. The statins are administered parenterally in a manner to provide the statin systemically, for an extended period, and to enhance the beneficial effect of statins for ameliorating cellular dysfunction, such as aberrant cellular proliferation, e.g. neoplasia and hyperplasia, cognitive dysfunction and pulmonary hypertension. Particularly, statins are provided other than orally, whereby the statins are maintained in the blood stream and associated with the dysfunctional tissue for extended periods of time at therapeutic dosage, with the exception of treatment of liver cancer, where statins may be administered orally at elevated dosages. Particularly, the statins are administered transdermally, by injection, employing particles or by infusion. The statins are used independently of other drugs or as a component of a therapeutic regimen.

### BRIEF DESCRIPTION OF THE FIGURES

FIGS. 1A and 1B, respectively, are graphs of the blood levels of lovastatin upon transdermal application (circles) and oral administration (inverted triangles) of lovastatin.

FIG. 2 is a graph of the pharmacokinetics of blood level with time of rabbit, dog and rat, with the rabbit treated intramuscular or oral with 5 mg/kg LS55 (2.75 mg/kgAPI) and the dog treated intramuscular or oral with a single administration of LS100, where using the closed or open form of lovastatin is differentiated. Dashed lines were hand drawn between non-consecutive points.

FIG. 3 is a graph of the pharmacokinetics for rabbit and dog as described in FIG. 2 on a log scale.

### DESCRIPTION OF THE SPECIFIC EMBODIMENTS

Effective treatment of cellular dysfunction is achieved with statin treatment under parenteral conditions where the statins are maintained at a therapeutic dosage for extended periods of time. The treatment finds application in such dysfunctions as aberrant cellular proliferation, such as hyperplasia and neoplasia, e.g. cancer, cognitive dysfunction, e.g. Alzheimer disease, and pulmonary hypertension. For the most part, the statins are administered to provide sustained therapeutic levels in proximity to the dysfunctional tissue over extended periods of time without being largely sequestered in a specific organ, e.g. liver, as results from oral administration. Of particular interest is the use of transdermal administration, injection, employing particles, or infusion, where as contrasted with oral administration, direct transport and accumulation of the statins does not occur in the liver. By using the subject methods of administration, the statins, by themselves or in conjunction with other therapeutic agents result in amelioration, usually improvement, in bodily function and extension of survival periods for the host, normally human. By amelioration is intended an improved outcome as compared to the absence of treatment with statins, such as reduced progress of the dysfunction, remission, reduced suffering from the dysfunction and cure.

Parenteral administration provides for the extended presence of the statin in the blood stream at a therapeutic level, so as to continuously act on the dysfunction for an extended period of time without interruption associated with periodic administration. The subject method of administration is more convenient and ensures more efficient and effective treatment of the indicated dysfunctions.

In addition, statins may be administered orally for the treatment of liver cancer, where super doses are employed exceeding 80 mg/d, the maximum dosage used for reducing cholesterol levels. Generally, the dosage will be greater than 80 mg/d, usually greater than about 100 mg/d and not more than about 200 mg/d, usually not more than about 160 mg/d. The treatment will generally be for at least 3 d, more usually at least about 5 d, and may be for 30 d or more.

For the most part, the modes of administration are parenteral and include transdermal administration, injection of the drug in an appropriate form and medium, administration by a pump, and the like. The statin HMG Co-A reductase inhibitors may be present in a fluid medium, solvent or non-solvent, dissolved or stably dispersed, as particles, where the particles may vary from 5 to 100% of the statin therapeutic agent, dispersed neat or as particles in a gel, e.g. hydrogel or temperature sensitive gel, impregnated, coated or formed as a film, mesh or fiber, normally in conjunction with a carrier, particularly a polymer matrix, and the like. It is desirable that the method provide a reservoir of the statin therapeutic agent to allow for continued administration of the statin at a therapeutic level to the dysfunctional tissue.

### General Considerations for Administration of HMG-coA Reductase Inhibitor

The parenteral mode of administration should provide a therapeutic amount of the HMG Co-A reductase inhibitor for sufficient time to provide the desired therapy, e.g. reduction or remission of the tumor being treated, enhanced cognitive function or reduced rate of loss of cognitive function. The amount of the HMG Co-A reductase inhibitors is the bioavailable amount, as drug that is not available to the site of interest, e.g. sequestered by an organ or subject to rapid degradation, will not provide the desired effect. Dosage levels will generally be in the range of about 0.01 to 3 mg/kg/day, more usually 0.1 to 2 mg/kg/day and frequently 0.1 to 1.5 mg/kg/day, where the amount may be modified to some degree depending upon the manner of administration, the choice of statin, the nature of the dysfunction, and the other drugs that are used in combination with the statin. In the case of cancer, other considerations include the history of the treatment of the tumor with statins and/or other drugs, the size of the tumor, the resistance of the tumor to the administered drugs, the prior response to the same or different treatment, and the like.

The treatment duration for humans will generally be greater than 1 day, usually greater than 2 days, more usually greater than about 5 days, desirably up to and including 10 days and not more than about 65 days, usually not more than about 25 days, and more usually not more than about 15 days, generally not more than 10 days. There may be cycles of treatment, where the patient may be treated for a relatively short period, e.g. 1 day, followed by a period of no treatment, e.g. 1 day to 1 month, and the treatment then repeated at the same or different dosage. Generally, the total dosage of the course of treatment will be at least about 1 mg, usually at least about 5 mg, and not more than about 5000 mg, usually not more than about 2000 mg, and frequently not more than about 500 mg. Treatment is terminated in the case of cancer when further treatment results in no further diminution of the tumor or experience indicates that there should be an extended period of observation to see whether the tumor continues to diminish in size or there has been total remission. For other indications, reduction, slowing of deterioration or stasis may be the determining factor.

Until there has been substantial use of the subject methodology, monitoring of the patient will be valuable to ascertain the optimum dosage and optimum duration. Once experience has been obtained with a specific formulation and particularly with a specific indication that experience may then be used in future therapies. In addition, since with cancer it has become common to use cocktails of drugs, the specific dosage and choice of statin will depend upon performing clinical trials to optimize the combinations for particular cancers, particular stages and particular patients. Insight into the amount of statins by themselves or in combination can be obtained by culturing cancer cells with the drugs of the therapeutic protocol at different levels of treatment and determining the response of the cancer cells to the different therapeutic regimens. One can also screen the cancer cells to determine the effect of the regimen on the expression of proteins related to the maintenance of the cancer. One can also study the effect of the regimen on angiogenesis by culturing epithelial cells associated with the tumor tissue. The cancer may be in any stage, e.g. Stages I, II, III or IV.

For indications other than cancer, a therapeutic dosage is administered for a limited period of time, from about 1 to 7 days, and depending upon the indication, physiological events are monitored, such as blood proteins, responses to tests, mental and/or physical abilities, etc., to determine whether the treatment is having a salutary effect as compared to the status of the patient prior to the treatment. As experience is gained, the dosage may be altered and responses further evaluated.

Various HMG-CoA reductase inhibitors may be used and as new HMG-CoA reductase inhibitors or their analogs are developed they are also included. Statins known today are described in S. E. Harris, et al. (1995) Mol Cell Differ 3, 137; G. Mundy, et al. Science (1999) 286, 1946; and U.S. Pat. Nos. 6,022,887; 6,080,779 and 6,376,476, whose disclosure of statins is specifically incorporated herein by reference. Illustrative statins include lovastatin, pravastatin, velostatin, simvastatin, fluvastatin, cerivastatin, mevastatin, dalvastatin, fluindostatin, rosuvastatin and atorvastatin. Also included are prodrugs of these statins, their pharmaceutically acceptable salts, e.g. calcium, etc. The preparation of these compounds is well known as set forth in numerous U.S. Pat. Nos. 3,983,149; 4,231,938; 4,346,227; 4,448,784; 4,450,171; 4,681,893; 4,739,073; and 5,177,080. Since these compounds are also generally commercially available, they can be purchased as required.

Any of the cancers or dysplasias leading to cancers can benefit from the treatment with statins. Common cancers include both solid cancers, such as carcinomas, sarcomas, lymphomas and melanomas and non-solid cancers, such as leukemias. Specific cancers include acute leukemia, anal cancer, bladder cancer, bone cancer, brain tumors, bowel cancer (colorectal cancer), breast cancer, cervical cancer, chronic lymphocytic leukemia (CLL) chronic myeloid leukemia (CML), choriocarcinoma, eye cancer, gallbladder cancer, gestational trophoblastic tumors (molar pregnancy and chriocarcinoma), head and neck cancer, Hodgkin's lymphoma, kidney cancer, larynx or laryngeal cancer (cancer of the voice box), leukemia, liver cancer, lung cancer, melanoma skin cancer, mesothelioma, mouth and oropharyngeal cancer, myeloma, nasal and paranasal sinus cancer, nasopharyngeal cancer, non-Hodgkin's lymphoma, oesophageal cancer, ovarian cancer, pancreatic cancer, penile cancer, prostate cancer, skin cancer (not melanoma), soft tissue sarcoma, stomach (gastric) cancer, testicular cancer, thyroid cancer, vaginal cancer, vulval cancer, womb cancer, gynaecological cancer. Brain tumors may be further divided into gliomas, meningiomas, pituitary adenomas, vestibular schwannomas, medulloblastomas, etc. Also included are neurofibromas.

Other antineoplastic drugs that can find use in combination with the statins fall into a number of cateogories. These include the nitrogen mustards: nitrosoureas, alkyl sulfonates, aziridines and platinum compounds; antimetabolites: folate antagonists, pyrimidine analogs, purine analogs, and sugar-modified analogs; non-covalent DNA binding drugs: anthracycline and bleomycin; inhibitors of chromatin function: topoisomerase inhibitors and microtubule inhibitors; drugs affecting endocrine function: tamoxifen. Specific drugs that can find use in conjunction with statins include cis-plat, doxorubicin, 5-FU, paclitaxel, ubiquinone, etoposide, tamoxifene, taxotere, adriamycin, azathioprine, bisulfan, casodex, cyclophosphamide, methotrexate, vincristine, imatinib, Rituximab®, and Herceptin®, as illustrative of the many anticancer drugs that find use individually or in combination. In use in accordance with this invention, their conventional dosages would be used and reduction in the amounts investigated by virtue of the combination with statins.

For non-cancerous dysfunctions, such as Alzheimer's disease, cognitive dysfunctions, and the like, the statins may be used by themselves or in conjunction with other drugs employed for the specific indication.

### Transdermal Application

In a preferred mode for providing the desired treatment as to concentration and duration, where one can achieve long term release while maintaining a relatively constant dosage to the site of interest, topical application can be employed.

As used herein, the phrase “topical application” describes application onto a biological surface, whereby the biological surface includes, for example, a skin area (e.g., hands, forearms, elbows, legs, face, nails, anus and genital areas) or a mucosal membrane. By selecting the appropriate carrier and optionally other ingredients that can be included in the composition, as is detailed herein below, the compositions of the present invention may be formulated into any form typically employed for topical application.

Hence, the pharmaceutical compositions of the present invention can be, for example, in a form of a cream, an ointment, a paste, a gel, a lotion, milk, a suspension, an aerosol, a spray, foam, a pad, and a patch.

Ointments are semisolid preparations, typically based on petrolatum or petroleum derivatives. The specific ointment base to be used is one that provides for optimum delivery for the active agent chosen for a given formulation, and, preferably, provides for other desired characteristics as well (e.g., emollience). As with other carriers or vehicles, an ointment base should be inert, stable, nonirritating and nonsensitizing. As explained in Remington: The Science and Practice of Pharmacy, 19th Ed., Easton, Pa.: Mack Publishing Co. (1995), pp. 1399-1404, ointment bases may be grouped in four classes: oleaginous bases; emulsifiable bases; emulsion bases; and water-soluble bases. Oleaginous ointment bases include, for example, vegetable oils, fats obtained from animals, and semisolid hydrocarbons obtained from petroleum. Emulsifiable ointment bases, also known as absorbent ointment bases, contain little or no water and include, for example, hydroxystearin sulfate, anhydrous lanolin and hydrophilic petrolatum. Emulsion ointment bases are either water-in-oil (W/O) emulsions or oil-in-water (O/W) emulsions, and include, for example, cetyl alcohol, glyceryl monostearate, lanolin and stearic acid. Preferred water-soluble ointment bases are prepared from polyethylene glycols of varying molecular weight.

Lotions are preparations that are to be applied to the skin surface without friction. Lotions are typically liquid or semiliquid preparations in which solid particles, including the active agent, are present in a water or alcohol base. Lotions are typically preferred for treating large body areas, due to the ease of applying a more fluid composition. Lotions are typically suspensions of solids, and oftentimes comprise a liquid oily emulsion of the oil-in-water type. It is generally necessary that the insoluble matter in a lotion be finely divided. Lotions typically contain suspending agents to produce better dispersions as well as compounds useful for localizing and holding the active agent in contact with the skin, such as methylcellulose, sodium carboxymethyl-cellulose, and the like.

Creams are viscous liquids or semisolid emulsions, either oil-in-water or water-in-oil. Cream bases are typically water-washable, and contain an oil phase, an emulsifier and an aqueous phase. The oil phase, also called the “internal” phase, is generally comprised of petrolatum and/or a fatty alcohol such as cetyl or stearyl alcohol. The aqueous phase typically, although not necessarily, exceeds the oil phase in volume, and generally contains a humectant. The emulsifier in a cream formulation is generally a nonionic, anionic, cationic or amphoteric surfactant. Reference may be made to Remington: The Science and Practice of Pharmacy, supra, for further information.

Pastes are semisolid dosage forms in which the bioactive agent is suspended in a suitable base. Depending on the nature of the base, pastes are divided between fatty pastes or those made from a single-phase aqueous gel. The base in a fatty paste is generally petrolatum, hydrophilic petrolatum and the like. The pastes made from single-phase aqueous gels generally incorporate carboxymethylcellulose or the like as a base. Additional reference may be made to Remington: The Science and Practice of Pharmacy, for further information.

Gel formulations are semisolid, suspension-type systems. Single-phase gels contain organic macromolecules distributed substantially uniformly throughout the carrier liquid, which is typically aqueous, but also, preferably, contain an alcohol and, optionally, an oil. Preferred organic macromolecules, i.e., gelling agents, are crosslinked acrylic acid polymers such as the family of carbomer polymers, e.g., carboxypolyalkylenes that may be obtained commercially under the trademark Carbopol™. Other types of preferred polymers in this context are hydrophilic polymers such as polyethylene oxides, polyoxyethylene-polyoxypropylene copolymers and polyvinylalcohol; modified cellulose, such as hydroxypropyl cellulose, hydroxyethyl cellulose, hydroxypropyl methylcellulose, hydroxypropyl methylcellulose phthalate, and methyl cellulose; gums such as tragacanth and xanthan gum; sodium alginate; and gelatin. In order to prepare a uniform gel, dispersing agents such as alcohol or glycerin can be added, or the gelling agent can be dispersed by trituration, mechanical mixing or stirring, or combinations thereof.

Sprays generally provide the active agent in an aqueous and/or alcoholic solution which can be misted onto the skin for delivery. Such sprays include those formulated to provide for concentration of the active agent solution at the site of administration following delivery, e.g., the spray solution can be primarily composed of alcohol or other like volatile liquid in which the active agent can be dissolved. Upon delivery to the skin, the carrier evaporates, leaving concentrated active agent at the site of administration.

Foam compositions are typically formulated in a single or multiple phase liquid form and housed in a suitable container, optionally together with a propellant which facilitates the expulsion of the composition from the container, thus transforming it into a foam upon application. Other foam forming techniques include, for example the “Bag-in-a-can” formulation technique. Compositions thus formulated typically contain a low-boiling hydrocarbon, e.g., isopropane. Application and agitation of such a composition at the body temperature cause the isopropane to vaporize and generate the foam, in a manner similar to a pressurized aerosol foaming system. Foams can be water-based or aqueous alkanolic, but are typically formulated with high alcohol content which, upon application to the skin of a user, quickly evaporates, driving the active ingredient through the upper skin layers to the site of treatment.

Skin patches typically comprise a backing, to which a reservoir containing the active agent is attached. The reservoir can be, for example, a pad in which the active agent or composition is dispersed or soaked, or a liquid reservoir. Patches typically further include a frontal water permeable adhesive, which adheres and secures the device to the treated region. Silicone rubbers with self-adhesiveness can alternatively be used. In both cases, a protective permeable layer can be used to protect the adhesive side of the patch prior to its use. Skin patches may further comprise a removable cover, which serves for protecting it upon storage.

Examples of patch configuration which can be utilized with the present invention include a single-layer or multi-layer drug-in-adhesive systems which are characterized by the inclusion of the drug directly within the skin-contacting adhesive. In such a transdermal patch design, the adhesive not only serves to affix the patch to the skin, but also serves as the formulation foundation, containing the drug and all the excipients under a single backing film. In the multi-layer drug-in-adhesive patch a membrane is disposed between two distinct drug-in-adhesive layers or multiple drug-in-adhesive layers are incorporated under a single backing film.

Another patch system configuration which can be used by the present invention is a reservoir transdermal system design which is characterized by the inclusion of a liquid compartment containing a drug solution or suspension separated from the release liner by a semi-permeable membrane and adhesive. The adhesive component of this patch system can either be incorporated as a continuous layer between the membrane and the release liner or in a concentric configuration around the membrane. Yet another patch system configuration which can be utilized by the present invention is a matrix system design which is characterized by the inclusion of a semisolid matrix containing a drug solution or suspension which is in direct contact with the release liner. The component responsible for skin adhesion is incorporated in an overlay and forms a concentric configuration around the semisolid matrix.

In all cases a cover or backing may be used to protect the formulation from abrasion or other kinds of removal. Any conventional cover may be used, such as dressings, plastic films, porous, breathing, or impermeable, tapes, etc.

Examples of pharmaceutically acceptable carriers that are suitable for pharmaceutical compositions for topical applications include carrier materials that are well-known for use in the cosmetic and medical arts as bases for e.g., emulsions, creams, aqueous solutions, oils, ointments, pastes, gels, lotions, milks, foams, suspensions, aerosols and the like, depending on the final form of the composition. Representative examples of suitable carriers according to the present invention therefore include, without limitation, water, liquid alcohols, liquid glycols, liquid polyalkylene glycols, liquid esters, liquid amides, liquid protein hydrolysates, liquid alkylated protein hydrolysates, liquid lanolin and lanolin derivatives, and like materials commonly employed in cosmetic and medicinal compositions. Other suitable carriers according to the present invention include, without limitation, alcohols, such as, for example, monohydric and polyhydric alcohols, e.g., ethanol, isopropanol, glycerol, sorbitol, 2-methoxyethanol, diethyleneglycol, ethylene glycol, hexyleneglycol, mannitol, and propylene glycol; ethers such as diethyl or dipropyl ether; polyethylene glycols and methoxypolyoxyethylenes (carbowaxes having molecular weight ranging from 200 to 20,000); polyoxyethylene glycerols, polyoxyethylene sorbitols, stearoyl diacetin, and the like.

Topical compositions of the present invention may, if desired, be presented in a pack or dispenser device, such as an FDA-approved kit, which may contain one or more unit dosage forms containing the active ingredient. The dispenser device may, for example, comprise a tube. The pack or dispenser device may be accompanied by instructions for administration. The pack or dispenser device may also be accompanied by a notice in a form prescribed by a governmental agency regulating the manufacture, use, or sale of pharmaceuticals, which notice is reflective of approval by the agency of the form of the compositions for human or veterinary administration. Such notice, for example, may include labeling approved by the U.S. Food and Drug Administration for prescription drugs or of an approved product insert. Compositions comprising the topical composition of the invention formulated in a pharmaceutically acceptable carrier may also be prepared, placed in an appropriate container, and labeled for treatment of an indicated condition.

The pharmaceutical composition of the present invention will be formulated to provide the indicated therapeutic level of HMG Co-A reductase inhibitor as indicated above. The amount of HMG Co-A reductase inhibitor may vary widely depending upon the specific formulation, the site at which the formulation is applied as compared to the site of interest requiring treatment, the area to which the formulation is applied, and the like. For the most part, the amount of the pharmaceutical composition ranges between about 1 mg and about 100 mg/cm² of the biological surface per day.

When provided as a cream or ointment, the pharmaceutical composition of the present invention typically includes HMG Co-A reductase inhibitor and a hydrophilic petrolatum, aqueous alkanolic gel or a pluronic lecithin organogel (PLO).

An aqueous alkanolic gel with a carbomer-based formulation can contain, for example, 60% ethanol, <40% ddH₂O, 1% Carbomer polymer of either 940 or 980, 0.5% cholesterol, 0.1% BHA, 3% TTA and HMG Co-A reductase inhibitor. Such a gel can be manufactured by slowly (drop wise) adding (while stirring) H₂O (1 ml) to a Carbomer 940/H₂O/triethanolamine mixture and slowly (drop wise) mixing in enough ethanol to make 10 ml of product. The pH of the final mixture should be >4.5. The final product is aliquotted and sealed and protected from light.

For pluronic gels selected components are combined and delivered in a topical vehicle, preferably pluronic lecithin organogel (PLO). Methods of topical application are as cream, gel, ointment, spray or patch, especially by iontophoresis delivering the components through an iontophoretic patch.

A preferred composition includes a HMG Co-A reductase inhibitor such as lovastatin and a topical gel preparation. The selected HMG Co-A reductase inhibitor is incorporated into pluronic lecithin organogel (PLO) to facilitate transdermal administration.

These components are mixed in a controlled environment. Precautionary measures should protect pharmaceutical workers from active ingredients that may become airborne or topically absorbable. In the United States, OSHA complaint safety procedures should be followed.

The composition can include a pharmaceutically acceptable liquid carrier which includes a biphasic complex of lecithin and organogel, for molecular egression across the epidermis to the superficial and deep dermis where vascular structures reside.

PLO is a phospholipid liposomal micro emulsion used for transdermal drug administration. PLO has two phases:

(i) An oil Phase: the oil phase is lecithin/isopropyl palmitate solution. Lecithin rearranges the horny layer of the skin. Isopropyl palmitate is a solvent and penetration enhancer. Sorbic acid is a preservative.

(ii) A water Phase: the water phase is a pluronic gel. Pluronic f127 NF is a commercial surfactant. Potassium sorbate NF is a preservative. Purified water is a solvent. The active agents are incorporated into the PLO gel and a stable emulsion is formed through sheer force. The concentration of the active agents in the formulation may be adjusted as to obtain the optimal therapeutic response.

A composition of the active agents and carrier is prepared according to the following procedure. First, HMG Co-A reductase inhibitor is solubilized; it is then combined with the lecithin/isopropyl palmitate solution and mixed well. Pluronic F127 is then added as a 20% gel in small increments to a final desired volume. The composition is then mixed at high speed in an electric mortar and pestle to form a smooth creamy gel.

Once prepared, the topical HMG Co-A reductase inhibitor formulation of the present invention can be administered topically either by the patient or by a heath care provider. When the dosage form is a topical cream-gel suspension or topical patch methodology, it may contain preservatives, stabilizers, emulsifiers or suspending agents, wetting agents, salts for osmotic pressure or buffers, as required. When the dosage form is as a pressurized spray or aerosol, the solution is contained in a pressurized container with a liquid propellant such as dichlorodifluororo methane or chlorotrifluoro ethylene. If administered from a pump container, the solution will include a buffer salt solution with preservatives, stabilizers, emulsifiers or suspending agents, wetting agents, and salts for osmotic pressure or buffers, as required.

When the composition is administered in the form of topical gel-cream, spray, or topical iontophoresis gel patch, the time of repeat application will vary from every six to twelve hours for the gel-cream and spray to several days for the topical iontophoresis gel-patch delivery methods. Occlusion with a barrier ointment or physical barrier such as hypoallergenic membrane may also be practiced after topical application of the gel-cream or spray to increase efficacy and penetration of the pharmaceutical.

When provided as a patch or any other transdermal delivery device, the pharmaceutical composition of the present invention includes a HMG Co-A reductase inhibitor, such as lovastatin. A preferred patch formulation would be a single-layer drug-in-adhesive system where the HMG Co-A reductase inhibitor in directly included within the skin-contacting adhesive. Preferred concentration ranges would be such that the patch delivers sufficient HMG Co-A reductase inhibitor for an effective concentration at the site of interest. Subject to the previously indicated caveats, this will generally fall within the ranges indicated above.

When provided as an aerosol or other transmucosal delivery device, the pharmaceutical composition of the present invention typically includes a HMG Co-A reductase inhibitor such as lovastastin. Preferred aerosol or other transmucosal delivery device would include technologies such as Metered Dose Inhalers (MDI) such as asthma inhalers which mediate the airways but not deep into the lungs, Nebulisers which would permit a fine liquid spray, dry Powder Inhalers (DPI) or liquid Micro proplet Inhalers. Alternative dosage forms for transmucosal or buccal delivery would include delivery systems such as mouthwashes, erodible/chewable buccal tablets, and chewing gums Bioadhesive buecut films/patches and tablets fabricated using various geometries either as a single-layer device, from which drug can be released multidirectionally or a device that has a impermeable backing layer on top of the drug-loaded bioadhesive layer where drug loss into oral cavity can be greatly decreased. Another device configuration can include a unidirectional release mechanism thus minimizing drug loss and enhancing drug penetration through the buccal mucosa.

Since HMG Co-A reductase inhibitors lower production of cholesterol which is a major component of cells including dermal and mucosal cells, topical administration of a HMG Co-A reductase inhibitor can lead to cholesterol depletion in such cells which could lead to reduced permeability of HMG Co-A reductase inhibitor. Thus, in order to increase the penetration of HMG Co-A reductase inhibitor through the biological surface, the pharmaceutical composition of the present invention preferably further includes cholesterol at a concentration of 0.1-5% by weight.

The pharmaceutical composition of the present invention can also include a penetration enhancer such as simple alkyl esters, phosopholipids, terpenes, supersaturated solutions, ultrasound, organic solvents, fatty acids and alcohols, detergents and surfactants, D-limonene, β-cyclodextrin, DMSO, polysorbates, bile acids, N-methylpyrrolidine, polyglycosylated glycerides, 1-dodecylazacycloheptan-2-one (Azone®), cyclopentadecalactone (CPE-215®), alkyl-2-(N,N-disubstituted amino)-alkanoate ester (NexAct®), 2-(n-nonyl)-1,3-dioxolane (SEPA®), Carbomer polymers, pluronic gels, lecithin, tri-block copolymers such as Pluronic 127 as well as stabilizers or neutralizers such as, BHA, benzoic acid, sodium hydroxide, potassium hydroxide triethanol Amine triethyl amine, other diluents in alkaline form, such as water, ethanol, and the like.

The present invention further encompasses processes for the preparation of the pharmaceutical compositions described above. These processes generally comprise admixing the active ingredients described hereinabove and the pharmaceutically acceptable carrier. In cases where other agents or active agents, as is detailed hereinabove, are present in the compositions, the process includes admixing these agents together with the active ingredients and the carrier. A variety of exemplary formulation techniques that are usable in the process of the present invention is described, for example, in Harry's Cosmeticology, Seventh Edition, Edited by J B Wilkinson and R J Moore, Longmann Scientific & Technical, 1982, Chapter 13 “The Manufacture of Cosmetics” pages 757-799 as well as in Pharmaceutical development and clinical effectiveness of a novel gel technology for transdermal drug delivery Alberti, I. et al Expert Opinions in Drug Delivery 2: 935-50, 2005, Mucosal drug delivery: membranes, methodologies, and applications, Song, Y et al Critical Reviews Therapeutic Drug Carrier Systems 21: 195-256, 2004 and Drug delivery systems: past, present, and future Mainardes, R. M. et al. Current Drug Targets 5: 449-55, 2004.

### Particle Administration

One form of HMG Co-A reductase inhibitors of particular interest is in the form of small particles, particularly micro- or nanoparticles. The compositions comprise particles that as a result of the low solubility of statins in aqueous media dissolve over time or slow release particles, nano or micro, comprising at least one HMG-CoA reductase inhibitor. The particles can be formed in any convenient manner to provide for homogeneous, substantially homogeneous or heterogeneous size distribution. In one aspect, the particles are administered to a site of interest in an appropriate vehicle and maintained at the site of interest for sufficient time to provide therapeutic levels. Generally, the particles will release the HMG-CoA reductase inhibitor at a rate as indicated above. For cancer or other localized indication, by site of interest is intended the site of a solid tumor, generally being within about 5 cm of the site, particularly upstream of the site, so as to release the HMG-CoA reductase inhibitor directly in association with the tumor being treated. However, there can be instances where the particles will be administered at a different site and the effect will rely on the release of the HMG-CoA reductase inhibitor from the particles where the released HMG-CoA reductase inhibitor is transported to the site of interest. With indications other than cancer, normally the particles will be placed in proximity to the diseased tissue or at a site where the blood flow carries the statins from the particles to the site of the dysfunction. Where appropriate the particles can be injected directly into the tumor to localize release of statin

The particles provide for a continuing therapeutic amount of the HMG-CoA reductase inhibitor over the prescribed treatment period. The particles administered provide for a relatively uniform release of the HMG-CoA reductase inhibitor over a predetermined period of time. By appropriate selection of particle composition and amount of particles administered, the period of time at which the site of interest is exposed to the drug at a therapeutic level provides for controlled therapeutic treatment. The particles are prepared to allow for the slow release of the HMG-CoA reductase inhibitor at a predetermined rate, so that over the period of treatment, the level of HMG-CoA reductase inhibitor at the site is sufficient to provide cell death. The particles may vary from substantially homogeneous HMG-CoA reductase inhibitor, as pure drug particles, varying from completely crystalline to completely amorphous and/or vitrified, to particles with the HMG-CoA reductase inhibitor as small particles interspersed in a carrier, a single core, or HMG-CoA reductase inhibitor molecules dispersed in a carrier, such as a hydrogel, which may include a rate controlling surface membrane.

The release of the HMG-CoA reductase inhibitor from the particles is controlled by non-mechanical means, namely physical and/or chemical phenomena. These phenomena include osmosis, dissolution, hydrolysis, degradation, solvation, erosion, etc. where the HMG-CoA reductase inhibitor is slowly released into the environment of the site of interest. Normally there is a curve where the release of the MHG-CoA reductase inhibitor is high and slowly decreases with time in a pseudo first order manner which is consistent with dissolution kinetics based upon the surface area of the particles. The release characterisitics can be altered by changing the shapes of the particles to increase the surface area and consequently increase the release rate of the drug. A cylindrical particle will therefore have a higher release rate and shorter duration of release than will a spherical particle. In such cases the particle remains intact during the course of the drug delivery. In yet other cases, there is a curve where initially the amount of HMG-CoA reductase inhibitor released increases to a maximum, followed by a low diminution of the amount of HMG-CoA reductase inhibitor released per unit time interval, and then frequently there is a breakdown of the particle where the remaining HMG-CoA reductase inhibitor is released over a short period of time. The average release rate will usually be between about 0.5 to 20%, more usually between about 5 to 20% to breakdown of the particles, based on a 24 h time period. Desirably, the residue at breakdown will be less that 20% of the original amount of HMG-CoA reductase inhibitor, preferably less than about 15%.

Depending upon the nature of the particles and the manner of their formation, one may have a substantially homogeneous sized composition of particles or a heterogeneous sized composition of particles, where the different sized particles will have different release profiles over time to provide the desired range of HMG-CoA reductase inhibitor concentration over the therapeutic time interval. The size dispersion may have two or more groups of sized particles, where each group will have at least about 75 weight % of particles of a size within 50% of the median size. Alternatively, one may have a relatively uniform narrow range or broad range of particle sizes.

The particles are biocompatible and conveniently bioresorbable, where particles comprising a carrier will normally be biodegradable. The particles will usually leave no residue and, if desired, will result in minimal inflammation, at the site being treated. At least 60 weight %, more usually at least about 70 weight % of the particles will be in the size range of about 0.001 to 100 μm, and generally at least about 60 weight %, more usually at least about 75 weight % will be within about 35%, preferably within about 20% of the median size particle for a homogeneous sized composition. (In referring to size one is considering the mean diameter.) Where the solid drug is milled or ground, one will usually have a heterogeneous mixture of particles where more than 50 weight %, more usually more than 60 weight %, will be within 50% of the median size of the particles. If desired, the particles may be sized using sieves or other method for providing particles in a particular range, where only particles in the particular range are used, or combinations of particles of the different ranges may be used. For a heterogeneous composition, there may be 1, 2 or 3 different groups having narrow size ranges, where the median size of any one group will usually be not more than about 100 times the next smaller median size, more usually not more than about 50 times the next smaller median size. The weight ratio of the groups will depend upon the release profile, where the smaller particles will generally release more of the HMG-CoA reductase inhibitor in the early period, while the larger particles will release the HMG-CoA reductase inhibitor later than the smaller particles.

In other cases it may be desirable to have more uniform particles. In such cases the particles can be prepared into nearly spherical form through a process such as an emulsification process. In such a process the drug of choice is dissolved into a solvent which is insoluble in water. Typically the drug is dissolved at high concentration in the solvent. Useful ranges for concentrations are on the order of 1 to 20%, more usually 5 to 15%, and 10% is frequently applicable. This solution is added with vigorous stirring to an aqueous solution containing a stabilizing agent to allow for stable formation of solvent droplets within the aqueous phase. Compounds such as the Tweens, e.g. Tween-80, can be used with good effect, but many other materials, e.g. non-ionic detergents, can be employed as known to those skilled in the art. During the course of mixing the solvent slowly dissolves into the water allowing for the formation of the spherical particle. The particles are further soaked in fresh water to remove adherent stabilizing agent and to further remove solvent. Drying these particles yields spherical particles whose size and size distribution are determined by the parameters used in the preparation process.

Spherical particles may also be prepared by other means including: homogeneous precipitation where the drug is dissolved in a solvent that is soluble in water which is then added to water with stirring; spray drying where the drug is dissolved into a volatile solvent which is then sprayed into an inert atmosphere at atmospheric or reduced pressure; melting the drug and dropping the melt through a sieve and allowing the droplets to cool while falling, to name but a few. One may use nanoparticles or microparticles, which may involve a carrier, where these groups of particles will fall into different size ranges. The nanoparticles will generally be in the range of about 1 to 50, more usually 5 to 25 nm, with the distribution as indicated above. The microparticles will generally be in the range of about 1 to 200 μm, more usually in the range of about 5 to 100 μm, with the distribution as indicated above. Only a few large particles can unduly distort the weight/size distribution. It should be understood that in the event of a few outliers the numbers given may be somewhat off and such outliers should not be considered in the distribution, as they generally will not exceed 10 weight % of the composition and will be at least about 1.5 times greater than the largest particle coming within the distribution.

The particle composition will be chosen to provide a continuous level of HMG-CoA reductase inhibitor at the site of interest, based on the area of the site to be treated, providing levels of application as described previously. More than one injection may be involved, so that the particle composition provides for the predetermined duration. The total number of days has been indicated previously. Where successive injections are employed, there may be periods of overlap, where the total amount of HMG-CoA reductase inhibitor being released for a short period, generally less than about 12 hours, more usually less than about 6 hours, is in excess of the amount indicated above. In order to achieve extended lengths of time while maintaining a therapeutic level, one or more administrations of the particles may be required, usually not more than daily and preferably not more than at intervals of about 3 days, more usually not more than at intervals of about 7 days, desirably at intervals not more than about 10 days, and may be single doses at intervals of 30 or more days.

The HMG-CoA reductase inhibitor can be prepared neat as a vitreous or crystalline particle. The particles can be either micro or nano as the sizes have been described above, and may be amorphous or crystalline, where the crystallinity can vary from about 0 to 100%. For slower release, the at least substantially crystalline particles will be used, where for more rapid release more of the amorphous drug will be present. One may also use powders where the pure drug is milled or ground to a predetermined size distribution. Various mechanical methods may be employed to provide the desired powder size distribution. Generally, large clumps are avoided, so that a relatively narrow size distribution is obtained, conveniently falling within the size range of the nano- or microparticles, but may also include fines that may fall outside those ranges. The fines will generally be less than about 20, usually less than about 10 weight % of the composition.

A wide range of particle compositions may be employed depending upon the nature of the site to be treated, the desired release profile, the amount of HMG-CoA reductase inhibitor required for the treatment, the time interval for providing the therapeutic level of HMG-CoA reductase inhibitor and the permitted volume of the particles at the site of interest.

One or more compositions may be used in the particle matrix, where one composition may be dispersed in the other, form a partial or complete coating of the other composition, or the like and the HMG-CoA reductase inhibitor may be an internal particle, e.g. core, or dispersed in one or more of the compositions to provide the desired slow release profile. The polymers that find use include both addition polymers and condensation polymers. The polymeric compositions that find use are biocompatible polymers that are normally resorbable, particularly biodegradable, which biodegradable polymers include: polymers of water soluble hydroxylaliphatic acids, particularly α-hydroxyaliphatic acids, oxiranes, vinyl compounds, urea derivatives, saccharides, orthoesters, anhydrides, hydrogels, etc. Compositions that may find use include polylactic acid (PLA) either a pure optical isomer or mixture of isomers, polyglycolic acid (PGA), copolymers of lactic acid and its optically active forms and glycolic acid (PGLA), copolymers of lactic acid and caprolactone, copolymers of glycolic acid and caprolactone, terpolymers of lactic acid, glycolic acid and caprolactone, polycaprolactone; polyhydroxybutyrate-polyhydroxyvalerate copolymer; poly(lactide-co-caprolactone); polyesteramides; polyorthoesters; poly co-hydroxybutyric acid; and polyanhydrides, block copolymers of the preceding with poly(ethylene glycol), or block copolymers of any combination of the preceding polymers.

Polymers which are generally biocompatible but not biodegradable include polymers such as: polydienes such as polybutadiene; polyalkenes such as polyethylene or polypropylene; polymethacrylics such as polymethyl methacrylate or polyhydroxyethyl methacrylate; polyvinyl ethers; polyvinyl alcohols; polyvinyl chlorides; polyvinyl esters such as polyvinyl acetate; polystyrene; polycarbonates; poly esters; cellulose ethers such as methyl cellulose, hydroxyethyl cellulose or hydroxypropyl methyl cellulose; cellulose esters such as cellulose acetate or cellulose acetate butyrate; polysaccharides; and starches, alkyl cyanoacrylates, polyurethanes.

Crosslinked biocompatible but not biodegradable polymers include hydrogels prepared from polyvinyl acetate (PVA), polyvinyl pyrrolidone, polyvinyl alcohol (xl-PValc), polyalkyleneoxides, particularly polyethylene oxide (PEG), etc., where the polymers may be cross-linked, modified with various groups, such as aliphatic acids of from 2 to 18 carbon atoms, alkyleneoxy groups of from 2 to 3 carbon atoms, and the like. The polymers may be homopolymers, co-polymers, block or random, may include dendrimers, etc.

Of particular interest are the polymers and copolymers of α-hydroxyaliphatic carboxylic acids of from 2-3 carbon atoms. Lactide/glycolide polymers for drug-delivery formulations are typically made by melt polymerization through the ring opening of lactide and glycolide monomers. Some polymers are available with or without carboxylic acid end groups. When the end group of the poly(lactide-co-glycolide), poly(lactide), or poly(glycolide) is not a carboxylic acid, for example, an ester, then the resultant polymer is referred to herein as blocked or capped. The unblocked polymer, conversely, has a terminal carboxylic group. The biodegradable polymers herein can be blocked or unblocked. In a further aspect, linear lactide/glycolide polymers are used; however star polymers can be used as well. Low or medium molecular weight polymers are used for drug-delivery where resorption time of the polymer and not material strength is important. The lactide portion of the polymer has an asymmetric carbon. Commercially racemic DL-, L-, and D-polymers are available. The L-polymers are more crystalline and resorb slower than DL-polymers. In addition to copolymers comprising glycolide and DL-lactide or L-lactide, copolymers of L-lactide and DL-lactide are available. Additionally, homopolymers of lactide or glycolide are available.

In the case when the biodegradable polymer is, poly(lactide), poly(glycolide), or poly(lactide-co-glycolide), in the latter case the amount of lactide and glycolide in the polymer can vary. In a further aspect, the biodegradable polymer contains 0 to 100 mole %, 40 to 100 mole %, 50 to 100 mole %, 60 to 100 mole %, 70 to 100 mole %, or 80 to 100 mole % lactide and from 0 to 100 mole %, 0 to 60 mole %, 10 to 40 mole %, 20 to 40 mole %, or 30 to 40 mole % glycolide, wherein the amount of lactide and glycolide is 100 mole %. In a further aspect, the biodegradable polymer can be poly(lactide), 95:5 poly(lactide-co-glycolide) 85:15 poly(lactide-co-glycolide), 75:25 poly(lactide-co-glycolide), 65:35 poly(lactide-co-glycolide), or 50:50 poly(lactide-co-glycolide) where the ratios are mole ratios.

Polymers that are useful for the present invention are those having an intrinsic viscosity of from 0.15 to 2.0, 0.15 to 1.5 dL/g, 0.25 to 1.5 dL/g, 0.25 to 1.0 dL/g, 0.25 to 0.8 dL/g, 0.25 to 0.6 dL/g, or 0.25 to 0.4 dL/g as measured in chloroform at a concentration of 0.5 g/dL at 30° C. In a further aspect, when the biodegradable polymer is poly(lactide-co-glycolide), poly(lactide), or poly(glycolide), the polymer has an intrinsic viscosity of from 0.15 to 2.0, 0.15 to 1.5 dL/g, 0.25 to 1.5 dL/g, 0.25 to 1.0 dL/g, 0.25 to 0.8 dL/g, 0.25 to 0.6 dL/g, or 0.25 to 0.4 dL/g as measured in chloroform at a concentration of 0.5 g/dL at 30° C.

Other forms of particles may be used, such as a core coated with a mixture of the HMG-CoA reductase inhibitor and an adhesive or other polymeric matrix. For example, an inorganic core or an organic core, such as collagen or other protein, organic polymer, etc., in the form of fibers, mesh, etc. may be used.

Among gels, of particular interest are thermoreversible gels that at a lower temperature are readily flowable and injectable, while at an elevated temperature become more rigid. This can be achieved, for example with the dispersion of the HMG-CoA reductase in mucoadhesive compositions, such as Noveon, particularly combined with a thermosensitive material, such as Pluronic F-127. Exemplary compositions are described in Tirnaksiz and Robinson, Pharmazie 2005, 60(7):518-23. (This reference is specifically incorporated by reference in its entirety.)

Other gels that may be employed with good effect include those of biological origin and provide properties such as viscoelasticity and/or thixotropy. Examples of such materials include hyaluronic acid, chondroitin sulfate, xanthan gums, cellulose derivatives, such as hydroxypropylmethyl cellulose, and the like. These materials allow for the uniform suspension of particles and provide good biocompatibility and ease for injection of the particles.

Where the HMG-CoA reductase inhibitor is mixed with a matrix, the amount of HMG-CoA reductase inhibitor will usually not exceed 95 weight %, frequently not exceed 60%, more usually not exceed 50 weight %, and will usually be not less than about 10 weight %, more usually not less than about 20 weight %. (The particles may have other components, so that the weight percents are based on just the two components, the HMG-CoA reductase inhibitor(s) and the matrix.) Where more than one polymer is used, each polymer will be present in at least 1 weight % of the particle, more usually at least about 5 weight % of the particle. Of course, polymer coatings that may be applied for numerous different reasons may be less than 1%, where the polymer coating serves to enhance the mechanical integrity of the particles, reduce abrasion, reduce deliquescence or efflorescence, ease of handling and flowing, control the rate at which the drug is released from the particle, etc.

The weight ratio of HMG-CoA reductase inhibitor to polymer will be in the range of about 0.1-20:1, more usually in the range of about 0.25-1.5:1, being consistent with the percentages indicated above.

The number of particle compositions and methods of preparation of particles are legion. Illustrative patents and patent applications include U.S. Pat. Nos. 4,687,660; 5,128,798; 5,427,798; and 6,510,430 and U.S. application nos. 2005/0165203; 0208134; 0255165; 0287114; 0287196; and 2006/0057222, and references cited therein. Textbooks that describe the considerations in selecting the compositions and preparing the particles include: Organic Chemistry of Drug Design and Drug Action, Richard B. Silverman, 1992; Drug Delivery: Engineering Principles for Drug Therapy, W. Mark Salzman, 2001 and Pharmacokinetics and Metabolism in Drug Design (Methods and Principles in Medicinal Chemistry) Dennis A. Smith, et al., 2001.

For the most part, the HMG-CoA reductase inhibitor and polymer matrix will be mixed together, usually in the presence of a solvent. Dropwise addition of the HMG-CoA reductase inhibitor to the matrix material may be used. After removing the solvent, the particles may be washed and sized. Other additives that may be used in the preparation of the particles include detergents, particular polymeric detergents, such as poly(vinyl alcohol/acetate)-partially hydrolyzed, e.g. 4-90 mol percent.

The particles can be used as a flowable mixture in a low viscosity medium, may be sintered or agglomerated to be formed into a porous mass or form, which may be further formed depending upon the site at which the particles are to be applied, may be introduced into bone cement materials, or the like. The particles can be joined to form the porous mass or form in a variety of ways. Partial solvents or softening agents may be used that soften the particle matrix, resulting in the particles becoming joined. Conveniently, the particles may be packed in a vessel or container providing a desired form or provide a form that can be further modified and the partial solvent passed through the packing to soften the surfaces of the particles. The particles are then repeatedly washed with a non-solvent in which the partial solvent is soluble to remove the partial solvent and recreate the solid surface of the particles. Alternatively, the particles may be sintered at a mild temperature, generally under 60° C. whereby the surface is softened and the particles become joined.

The particles may be formed into the porous mass by themselves or in conjunction with other materials, that are conveniently of the size range indicated for the HMG-CoA reductase inhibitor particles and have the appropriate properties for forming the porous mass, e.g. having a composition or polymeric matrix the same as or responding in the same way to the treatment as the particles containing the HMG-CoA reductase inhibitor. Sintering conditions will depend to a substantial degree on the desired degree of porosity, the material(s) used for making the particles, the effect of sintering on the release of the HMG-CoA reductase inhibitor, and the like.

### Intravascular Infusion and Injection

The subject statins may be infused or injected, particularly in combination with one or more other drugs. Infusion can provide for systemic treatment, being intraarterially or intravenously. Injection may provide for local or systemic treatment. For cancer, the other drugs may be antiproliferation drugs, apoptotic inducing drugs, antiangiogenesis drugs, DNA binding drugs, antibodies, etc. A list of anticancer drugs may be found on the web page of the University of Maryland Medical Center under the title Anticancer Drugs, which disclosure is incorporated herein by reference. Any of these drugs can find use with statins in combination as part of a chemotherapeutic treatment. For other indications, as indicated previously, the statins may be used by themselves or in combination with drugs normally prescribed for the indication.

The statins can be formulated with pharmaceutically acceptable carriers that contain physiologically acceptable compounds that act, e.g., to stabilize the composition or to increase or decrease the absorption of the agent and/or pharmaceutical composition. Physiologically acceptable compounds can include, for example, carbohydrates, such as glucose, sucrose, or dextrans, antioxidants, such as ascorbic acid or glutathione, chelating agents, low molecular weight proteins, compositions that reduce the clearance or hydrolysis of the anti-cancer agents, or other stabilizers and/or buffers. Detergents can also used to stabilize the composition or to increase or decrease the absorption of the pharmaceutical composition.

Other physiologically acceptable compounds include wetting agents, emulsifying agents, dispersing agents or preservatives which are particularly useful for preventing the growth or action of microorganisms. Various preservatives are well known and include, for example, phenol and ascorbic acid. One skilled in the art would appreciate that the choice of a pharmaceutically acceptable carrier and adjuvants will be chosen in accordance with the particular active compounds in the formulation and the particular cancer being treated.

The compositions for administration will commonly comprise a dispersion, e.g. solution or suspension, of the anticancer agents dissolved in a pharmaceutically acceptable carrier, preferably an aqueous carrier, where detergents or physiologically acceptable organic solvents may be added to provide for stability. A variety of carriers can be used, e.g., buffered saline and the like. These solutions are sterile and generally free of undesirable matter. These compositions may be sterilized by conventional, well known sterilization techniques. The compositions may contain pharmaceutically acceptable auxiliary substances as required to approximate physiological conditions, such as pH adjusting and buffering agents, salts, and the like, for example, sodium acetate, sodium chloride, potassium chloride, calcium chloride, sodium lactate and the like. The concentrations of the various anticancer agents in these formulations can vary widely depending upon the selected drugs in the formulation, the nature of the treatment, the nature of the cancer, the response of the patient, and the like.

### EXPERIMENTAL

Plasma concentrations of lovastatin equivalents after a single dose were measured at several time points using a modification of the well-described HMG-CoA reductase inhibition assay [Germershausen J I, Hunt V M, Bostedor R G, Bailey P J, Karkas J D, Alberts A W (1989) Tissue selectivity of the cholesterol-lowering agents lovastatin, simvastatin and pravastatin in rats in vivo. Biochem Biophys Res Commun 158: 667-675.]. The soluble rat liver HMG-CoA reductase used in this assay was prepared from rat liver microsomes [Heller R A, Gould R G (1973) Solubilization and practical purification of hepatic 3-hydroxy-3-methylglutaryl coenzyme a reductase. Biochem Biophys Res Commun: 50: 859-865.]. Plasma was withdrawn from the rats after a single dose of lovastatin administered orally or dermally at 1, 3, 6 and 24 hours. The concentration of the drug was determined by comparing the amount of inhibitory activity in the plasma of treated rats to a standard curve generated by adding the active open ring form of lovastatin to normal rat plasma. This is a standard method of studying the pharmacokinetics/pharmacodynamics of lovastatin because this drug reportedly has several active metabolites. The area under the plasma concentration-time curve (AUC₀₋₂₄ₕᵣ) of lovastatin equivalence was calculated using the trapezoidal rule for both oral and dermal application of lovastatin. For oral administration, a suspension of lovastatin was prepared in 0.5% methylcellulose and administered by gavage. For dermal administration, lovastatin was mixed with hydrophilic petrolatum and applied to the back of the animals after shaving (area of application=6.45 cm²).

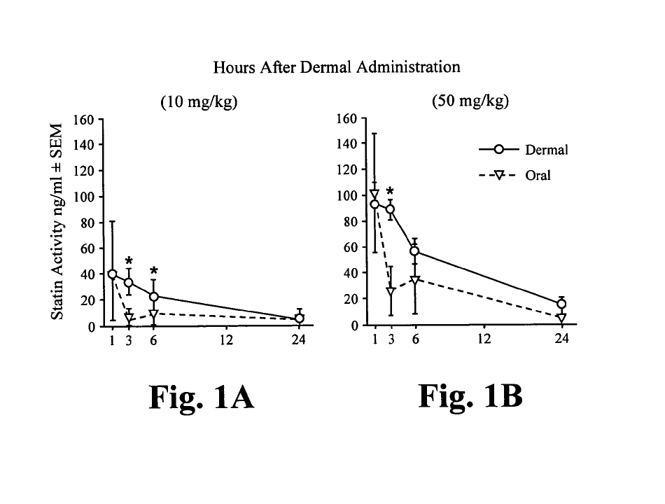

FIGS. 1A and 1B show plasma lovastatin levels of intact rats after a single dose of lovastatin administered orally or dermally at 1, 3, 6 and 24 hours. The level of the drug was determined as described above. Oral lovastatin was administered by gavage in 0.5% methylcellulose. For comparison, lovastatin was given dermally with application to the back of rats after shaving, using 100% DMSO as vehicle. Two different doses of lovastatin were administered as shown in panels A and B. Dermal application of lovastatin led to plasma concentrations of lovastatin which were greater, less variable and more prolonged than when the drug was given orally. Similar results were obtained with dermal application of lovastatin when hydrophilic petrolatum was substituted for DMSO as vehicle (data not shown). These results demonstrate the advantage of using parenteral administration to provide a stable amount of the statin in the blood over an extended period of time.

In another set of experiments the plasma level was determined for intramuscular injections of various formulations of lovastatin particles. In this study two different formulations were used, one designated LS55 was a composition containing 55% lovastatin in PLGA. This drug was prepared as spherical microparticles of nominal size of 70 microns by an emulsion process. The size of microspheres was selected based upon sieve cuts from the mixture obtained during the manufacturing process. Another drug form designated as LS100 consists of 100% lovastatin polycrystalline microspheres that were also prepared by an emulsion process and sieve cut to yield a mean particle diameter of 70 microns as determined by laser diffraction. The injection vehicle for all microparticles consisted of 75% phosphate buffered saline (Hyclone, USP grade) and 25% polyethylene glycol 400 (Spectrum Chemical Mfg. Corp., NF grade).

Lovastatin, once administered to an animal, can undergo hydrolysis of the lactone ring to generate the so called hydroxy acid form of the compound. The lactone form of the compound is referred to as the closed (c) form while the hydroxy acid is referred to as the open (o) ring form. This interconversion of forms is generally much higher in rodents owing to their higher blood esterase content as compared with higher mammals. Due to the conversion of the closed to the open ring forms in all species both the closed and open forms were tested by an LC/MS method.

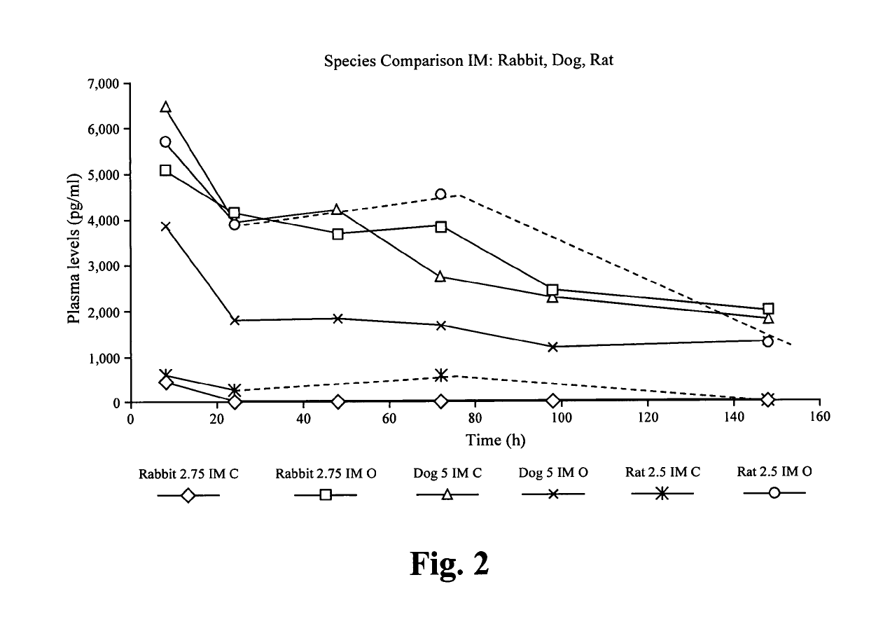

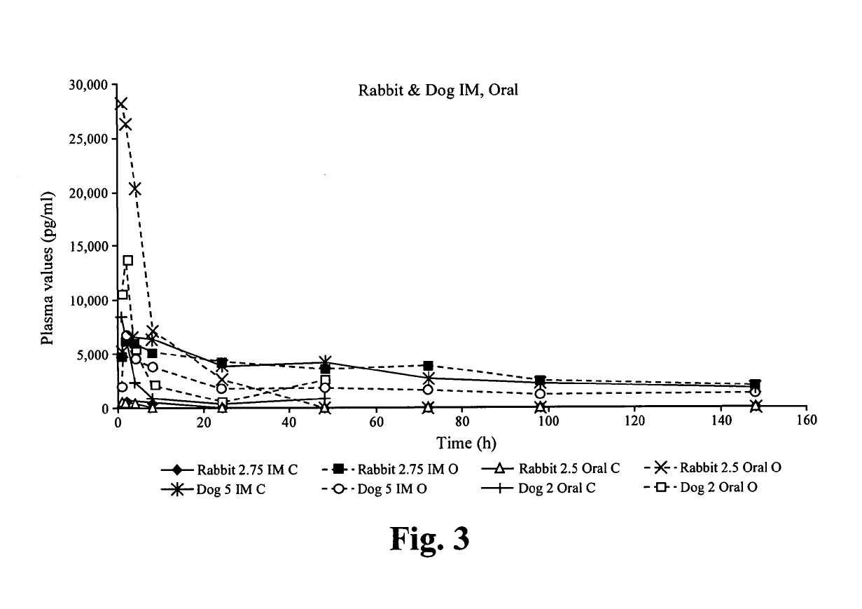

FIG. 2 shows the plasma levels for both the open and closed forms of lovastatin as a function of time in the dog, rat and rabbit after injection of either LS100 or LS55 drug products by intramuscular injection. FIG. 3 shows a comparison between intramuscular and oral administration of lovastatin in the dog and rabbit. In both figures the labels for each curve provide the following information: species, dose level, form of drug, route of administration and form of lovastatin. As is evident in the figures both the LS100 and LS55 provide a long term release of drug that decreases slowly with time.

In accordance with the subject invention cancer treatments are provided where statins are introduced at therapeutic concentrations to tumor containing patients diagnosed as requiring anticancer treatment. The statins provide an individual therapy, as well as an adjunct therapy in providing enhanced anticancer response, where in appropriate situations the other anticancer drugs that are frequently toxic to normal cells as well as cancer cells, can be successfully employed at reduced levels. By parenteral administration, e.g. intramuscular, subcutaneously, intravenously, transdermally, intratumorally, etc., substantial loss of the statins to the liver can be avoided so as to be able to maintain a therapeutic level in the vicinity of the tumor.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1. A method for treating a human patient diagnosed as having a physiological dysfunction selected from the group consisting of cancer, cognitive dysfunction and pulmonary hypertension, said method comprising:
parenterally administering to said human patient a therapeutic dosage of a statin for sufficient time to at least ameliorate said physiological dysfunction.

2. A method according to claim 1, wherein said administering is by injection.

3. A method according to claim 2, wherein said administering is by injection of statin containing statin releasing particles.

4. A method according to claim 2, wherein said injection is by infusion of statins.

5. A method according to claim 1, wherein said statins are administered transdermally.

6. A method according to claim 1, wherein said statins are administered at a rate of 0.01 to 3 mg/kg/day for at least one day.

7. A method for treating a human patient diagnosed as having cancer, said method comprising:
parenterally administering to said human patient a therapeutic dosage of a statin and at least one additional anticancer drug for sufficient time to inhibit the growth of said cancer.

8. A method according to claim 7, wherein said administering is by injection.

9. A method according to claim 8, wherein said administering is by injection of statin containing statin releasing particles.

10. A method according to claim 8 wherein injection is by infusion of statins.

11. A method according to claim 7, wherein said statins are administered transdermally.

12. A method according to claim 7, wherein said statins are administered at a rate of 0.01 to 3 mg/kg/day for at least one day.

13. A method for treating a human patient diagnosed as having cognitive dysfunction, said method comprising:
parenterally administering to said human patient a therapeutic dosage of a statin for sufficient time to change the progress of said cognitive dysfunction.

14. A method according to claim 13, wherein said statins are administered at a rate of 0.01 to 3 mg/kg/day for at least one day.

15. A method for treating a human patient diagnosed as having pulmonary hypertension, said method comprising:
parenterally administering to said human patient a therapeutic dosage of a statin for sufficient time to change the progress of said pulmonary hypertension.

16. A method according to claim 15, wherein said statins are administered at a rate of 0.01 to 3 mg/kg/day for at least one day.

17. A method for treating a human patient diagnosed as having liver cancer, said method comprising:
orally administering to said human patient a therapeutic dosage of a statin in excess of about 80 mgld for sufficient time to inhibit the growth of said liver cancer.

18. A method according to claim 17, wherein at least one additional anticancer drug is administered for sufficient time to inhibit the growth of said cancer.

19-22. (canceled)
